(19) United States
(12) Patent Application Publication
(10) Pub. No.: US 2010/0322983 A1
(43) Pub. Date: Dec. 23, 2010

(54) Personal-Care Composition

(76) Inventors: Susan Adair GRIFFITHS-BROPHY, Middletown, OH (US); Robert John Elsbrock, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/818,246

(22) Filed: Jun. 18, 2010

Related U.S. Application Data
(60) Provisional application No. 61/219,313, filed on Jun. 22, 2009.

(51) Int. Cl. A61K 8/06; A61Q 17/04; A61Q 19/00

(52) U.S. Cl. 424/401; 424/59; 514/18.8; 514/62; 424/600; 514/169; 514/563; 514/474; 514/636; 424/729

(57) ABSTRACT

A personal-care composition in the form of an oil-in-water emulsion is disclosed that provides improved skin feel and effective wrinkle-filling. The personal-care composition comprises a silicone elastomer; a silicone fluid; a combination of particulate materials; a polymeric thickener; and water. The combination comprises at least three particulate materials which pack together in order to fill wrinkles.

### CROSS REFERENCE TO RELATED APPLICATION

This application claims the benefit under 35 USC 119(e) to U.S. Application Ser. No. 61/219,313, filed Jun. 22, 2009.

### FIELD OF THE INVENTION

The present invention relates to personal-care compositions comprising three or more particle types for reducing the appearance of fine lines and wrinkles.

### BACKGROUND OF THE INVENTION

A variety of personal-care compositions are available to provide skin care benefits and to counteract what many consider undesirable “signs of skin aging,” such as fine lines, wrinkles, and uneven skin texture. To be most effective, compositions generally must be applied regularly and over an extended period of time. This may be especially important when the product is intended to provide a chronic, or long-term, benefit. To encourage frequent usage, it is important that the product have a desirable feel when applied. There exists a continuing need, therefore, to provide personal-care compositions that signal an immediate benefit of skin feel and thus encourage repeated use to provide a long-term benefit.

Silicone elastomers and silicone fluids have been used in consumer products to impart an improved skin feel as opposed to hydrocarbon-based products. Silicone elastomers and silicone fluids are typically described as providing a less heavy or greasy feel compared to hydrocarbon-based products. However, previous compositions have typically had a continuous silicone phase with a discontinuous aqueous phase. These water-in-silicone emulsions often exhibit problems with prolonged stability. Furthermore, silicone continuous phase products present processing complexities because water-in-silicone emulsions are typically sensitive to shear. The shear inherent in a large scale-processing line often may cause destabilization of these emulsions. Silicone continuous phases are further disfavored because processing lines after having conveyed silicone fluids are very difficult to clean without the use of solvents or a large quantity of surfactants.

Particulate material can be added to consumer products for a variety of reasons such as to improve the skin feel of the product. Particulate materials also may provide an immediate visible benefit to the skin by diffusely reflecting light, which provides a matting effect to the skin. However, many particulate materials are added to act as an opacifying agent, which effectively turns the consumer product into make-up and like products. These particulate materials have a tendency to exaggerate or emphasize fine lines and wrinkles. There is a need for a personal-care composition which reduces the noticability of fine lines and wrinkles. Specifically, there is a need for a personal-care composition which yields a noticeable reduction in the appearance of wrinkles by way of filling-in the wrinkles.

Therefore, a need exists for a personal-care composition providing the improved skin feel benefits by using silicone elastomers and a high percentage of silicone fluids without the adverse effects of a continuous silicone phase including the problems of stability, processing, and cleaning. A further need exists for the personal-care composition to comprise a high percentage of a particulate material without increasing the appearance of fine lines and wrinkles.

### SUMMARY OF THE INVENTION

In light of the needs presented above, the present invention relates to a personal-care composition in the form of an oil-in-water emulsion providing improved skin feel through the use silicone elastomers, a high percentage of silicone fluid, and a combination of particulate materials. The personal-care compositions are stable and have a silky, non-greasy feel. The particulate materials pack together to more effectively fill fine lines and wrinkles. In a particular embodiment, the present invention relates to a personal-care composition in the form of a oil-in-water emulsion having a silicone elastomer, a silicone fluid, a combination of particulate materials, a polymeric thickener, and water.

### DETAILED DESCRIPTION OF THE INVENTION

As used herein, the following terms shall have the meaning specified thereafter:

“Personal-care composition” means compositions suitable for topical application on mammalian keratinous tissue.

“Skin care actives,” or “actives,” as used herein, means compounds that, when applied to the skin, provide a benefit or improvement to the skin. It is to be understood that skin care actives are useful not only for application to skin, but also to hair, nails, and other mammalian keratinous tissue.

“Stable” and “stability” mean a composition which is substantially unaltered in chemical state, physical homogeneity, and/or color when the composition is at a temperature of from about 1° C. to about 40° C.

“Keratinous tissue” refers to keratin-containing layers disposed as the outermost protective covering of mammals which includes, but is not limited to, skin, hair, and nails.

“Regulating skin condition” means improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic or acute benefit and may include one or more of the following: reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

“Signs of skin aging,” include, but are not limited to, all outward visibly and tactilely perceptible manifestations, as well as any macro- or micro-effects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Non-volatile” means materials that exhibit a vapor pressure of no more than about 0.2 mm Hg at 25° C. at one atmosphere and/or to materials that have a boiling point at one atmosphere of at least about 300° C. “Volatile,” as used herein, all materials that are not “non-volatile” as defined herein.

“Safe and effective amount” means an amount of a compound or composition sufficient to induce a positive benefit but low enough to avoid serious side effects (i.e., provides a reasonable benefit to risk ratio within the judgment of a skilled artisan).

In all embodiments of the present invention, all percentages are by weight of the total composition, unless specifically stated otherwise. All ratios are weight ratios, unless specifically stated otherwise. All ranges are inclusive and combinable; therefore, every range given throughout this specification will include every narrower range that falls within such broader range as if such narrower ranges were all expressly written herein. The number of significant digits conveys neither a limitation on the indicated amounts nor on the accuracy of the measurements. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

### I. Personal-Care Composition

The personal-care composition of the present invention may be a skin care, cosmetic, or hair care product. The personal-care composition may be used as, for example, a moisturizer, conditioner, anti-aging compound, skin-lightening compound, sunscreen, or sunless tanner. In certain embodiments, the composition is applied to the face, neck, hands, arms, and other typically exposed areas of the body.

The compositions of the present invention are useful for improving skin appearance and/or feel. The compositions of the present invention may be useful for regulating skin condition and/or improving skin condition. In certain embodiments, the composition is useful for regulating and/or improving the signs of skin aging. The compositions may provide an essentially immediate (i.e., acute) improvement in skin appearance and/or feel following application. It is believed that the acute improvement may be attained with a single or limited number of applications of the composition. However, the compositions may comprise components that provide a gradual (i.e., chronic) improvement in skin appearance and/or feel. It is believed that the chronic improvement may involve multiple, reoccurring, or periodic applications of the composition. The compositions of the present invention may be incorporated into consumer products. In certain embodiments, the consumer products allow the composition to be applied as a spot treatment over a limited area of the skin.

In one embodiment, the compositions of the present invention yield a visibly noticeable reduction in wrinkles. This may be accomplished by an improvement in the way the composition fills-in wrinkles. A combination of particles packs together in a manner which effectively fills in wrinkles. A combination of at least three particulates is preferred. The particulates are of different sizes and/or shapes in order to gain the best packing action.

The compositions of the present invention are in the form of an emulsion. While various emulsion forms are known in the art (e.g., oil-in-water, water-in-oil, water-in-oil-in-water, and oil-in-water-in-oil emulsions), the compositions of the present invention are directed to an emulsion having a continuous aqueous phase and a discontinuous (i.e., internal) oil phase. The oil phase of the present invention comprises silicone oil (which may alternately called silicone fluids). However, the oil phase may also comprise non-silicone oils such as hydrocarbon oils, esters, ethers, and the like.

The aqueous phase typically comprises water. The composition may comprise about 8% to about 74% of water. Alternatively, the composition may comprise about 35% to about 60% of water. The aqueous phase may comprise components other than water (non-water components), including but not limited to water-soluble moisturizing agents, conditioning agents, anti-microbials, humectants and/or other water-soluble skin care actives, to impart an increased benefit to the keratinous tissue. In one embodiment, the non-water component of the composition comprises a humectant such as glycerin and/or other polyols.

A. Silicone Elastomer

The composition of the present invention comprises at least one silicone elastomer. Silicone elastomers are useful for reducing the tackiness of the composition and for providing a pleasant feel upon application. One non-limiting example of a suitable class of silicone elastomers is crosslinked organopolysiloxane (or siloxane) elastomers, which are generally described in U.S. patent application publication US2003/0049212A1. The crosslinked organopolysiloxane elastomers may be categorized as emulsifying or non-emulsifying. “Emulsifying,” as used herein, means crosslinked organopolysiloxane elastomers having at least one polyoxyalkylene (e.g., polyoxyethylene or polyoxypropylene) or polyglycerin moiety. The polyoxyalkylene (e.g., polyoxyethylene or polyoxypropylene) or polyglycerin moiety may serve as the crosslinker within the elastomer. Exemplary emulsifying crosslinked organopolysiloxane elastomers are described in U.S. Pat. Nos. 5,412,004; 5,837,793, and 5,811,487. “Non-emulsifying” means crosslinked organopolysiloxane elastomers are essentially free of polyoxyalkylene or polyglycerin moieties.

The composition of the present invention may comprise from about 0.5% to about 30% or, alternatively, from about 1% to about 20% of one or more silicone elastomers. The indicated percentages are understood to refer to amount of dry elastomer, as opposed to the total amount of elastomer and solvent, used for example for storage and shipping.

In certain embodiments, the composition of the present invention may comprise from about 0.5% to about 30% or, alternatively, from about 1% to about 20% of one or more non-emulsifying crosslinked siloxane elastomers. In these embodiments, the composition may comprise no to substantially no (i.e., less than 0.01%) emulsifying crosslinked siloxane elastomers. Exemplary non-emulsifying crosslinked siloxane elastomers include the CTFA (Cosmetic, Toiletry, and Fragrance Association International Cosmetic Ingredient Dictionary and Handbook, 11^{th} ed.) designated dimethicone/vinyl dimethicone crosspolymers supplied by a variety of suppliers including Dow Corning™ (DC 9506), General Electric™ (SFE 839), Shin Etsu™ (KSG 15 and 16), and Grant Industries (GRANSIL RPS-NA) and dimethicone/phenyl vinyl dimethicone crosspolymer such as KSG 18 available from Shin Etsu™. Other exemplary non-emulsifying crosslinked siloxane elastomer include the CTFA designated dimethicone crosspolymers including Dow Corning™ (DC 9040, DC 9041, DC 9045).

The composition of the present invention may comprise from about 0.5% to about 30% or, alternatively, from about 1% to about 20% of one or more emulsifying crosslinked siloxane elastomer such as described in U.S. Pat. Nos. 5,412,004; 5,837,793; and 5,811,487. Suitable emulsifying crosslinked organopolysiloxane elastomers include dimethicone/PEG-10 crosspolymers such as KSG 24; dimethicone/PEG-10 crosspolymers such as KSG 21 and KSG 210; PEG-15/lauryl dimethicone crosspolymers such as KSG 31, KG 32, KSG 33, KSG 310, KG 320, KSG 330; PEG-15/lauryl dimethicone crosspolymers and PEG-10/lauryl dimethicone crosspolymers such as KSG 34 and KSG 340; dimethicone/polyglycerine-3 crosspolymers such as KSG-710; and lauryl dimethicone/polyglycerine-3 crosspolymers such as KSG 810, KSG 820, KSG 830, and KSG 840. All KSG materials are available from Shin Etsu™

B. Silicone Fluid

The composition of the present invention comprises from about 0.5% to about 20%, or, alternatively, from about 1% to about 15% of one or more silicone fluids. In certain embodiments, the oil phase comprises a mixture of volatile silicones and/or non-volatile silicones. In certain embodiments, the silicone fluid may comprise from about 0.5% to about 20% volatile silicones.

Suitable volatile silicones include cyclic and linear volatile silicones. A description of various volatile silicones is found in Todd, et al. “Volatile Silicone Fluids for Cosmetics,” 91 Cosmetics and Toiletries 27-32 (1976). Suitable cyclic volatile silicones include cyclic dimethyl siloxane chains containing an average of from about 3 to about 9 silicon atoms, preferably from about 4 to about 6 silicon atoms. Exemplary cyclic volatile silicones of varying viscosities include Dow Corning DC 244, DC 245, DC 246, DC 344, and DC 345; GE Silicones-OSi Specialties Volatile Silicone 7207 and Volatile Silicone 7158; and GE Silicones SF1202. Suitable linear silicones include the polydimethylsiloxanes containing an average of from about 3 to about 9 silicon atoms. Exemplary linear volatile silicones include Dow Corning DC 200 and DC225 and the Shin Etsu DM-Fluid series. In certain embodiments, the linear volatile silicones generally have viscosities of less than or equal to about 6 centistokes at 25° C., and the cyclic materials generally have viscosities of less than about 10 centistokes at 25° C.

The nonvolatile silicones will have vapor pressures as previously defined. The nonvolatile silicones may exhibit an average viscosity of from about 10 to about 100,000 cps at 25° C. or from about 50 to about 10,000 cps. Examples of suitable nonvolatile silicones include, but are not limited polyalkylsiloxanes, polyarylsiloxanes, polyalkylarylsiloxanes, polyethersiloxane copolymers, and mixtures thereof. Examples of useful oils include the General Electric Viscasil™ series, General Electric SF1075, high viscosity members of the Dow Corning DC 200 series, and Dow Corning DC 556 Cosmetic Grade Fluid.

The personal-care composition may also comprise non-silicone oils. Suitable non-silicone oils include nonpolar, volatile hydrocarbon oils are discussed in Cosmetics, Science, and Technology, Vol. 1, 27-104 edited by Balsam and Sagarin, 1972. The nonpolar, volatile hydrocarbon oils may be saturated, unsaturated, aliphatic (straight or branched chains), alicyclic, or aromatic. Examples of preferred nonpolar, volatile hydrocarbon oils include, but are not limited to, isododecane (e.g., Permethyl-99A which is available from Presperse Inc.), isodecane, and the C7-C8 through C12-C15 isoparaffins (e.g., Isopar Series available from Exxon Chemicals).

Other suitable non-silicone oils include polar, nonvolatile oils such as fatty alcohols; fatty acids; esters of mono and dibasic carboxylic acids with mono and polyhydric alcohols; polyoxyethylenes; polyoxypropylenes; mixtures of polyoxyethylene and polyoxypropylene ethers of fatty alcohols; and mixtures thereof. The aforementioned oils may be saturated, unsaturated, aliphatic (straight or branched chains), alicyclic, or aromatic.

Other suitable non-silicone oils include nonpolar, nonvolatile hydrocarbon oils such as paraffinic hydrocarbon oils including mineral oils and certain branched-chain hydrocarbons as discussed in U.S. Pat. No. 5,019,375. Exemplary branched-chain hydrocarbons include Permethyl 103A (average of about 24 carbon atoms), Permethyl 104A (average of about 68 carbon atoms), and Permethyl 102A (average of about 20 carbon atoms); all may be purchased from Presperse, Inc., Somerset, N.J.

C. Particulate Material

The compositions of the present invention may comprise a combination of at least three particulate materials. The particulate material may be dispersed and suspended in the composition. The first particulate material may exhibit a median particle size of from about 5 μm to about 40 μm; from about 10 μm to about 30 μm; or, alternatively, from about 15 μm to about 25 μm. The second particulate material may exhibit a median particle size of from about 0.1 μm to about 20 μm; from about 0.5 μm to about 15 μm; or, alternatively, from about 1 μm to about 10 μm. The third particulate material may exhibit a median particle size of from about 0.1 μm to about 15 μm; from about 0.5 μm to about 10 μm; or, alternatively, from about 1 μm to about 5 μm. The composition of the present invention may further comprise a fourth particulate material. The fourth particulate material may exhibit a median particle size of from about 1 μm to about 25 μm; from about 3 μm to about 20 μm; or, alternatively, from about 5 μm to about 15 μm. Median particle size can be determined by any suitable method known in the art, such as by using coulter-counter equipment or the ASTM Designation E20-85 “Standard Practice for Particle Size Analysis of Particulate Substances in the Range of 0.2 to 75 Micrometers by Optical Microscopy,” ASTM Volume 14.02, 1993, incorporated herein by reference. The median particle size is measured when the particulate material is in the neat form (i.e., in the essentially pure, powder form prior to combination with the carrier of the invention).

In various embodiments, the particulates may be platelet shaped, spherical, elongated or needle-shaped, or irregularly shaped. In one embodiment, a mixture of spherical and platelet shaped particulates may be used to beneficially pack the particulates together in order to fill wrinkles. In another embodiment, a combination of different sized particulates may be used to enable functional packing of the particulates in order to fill wrinkles.

The compositions of the present invention preferably comprise from about 1% to about 15%, or from about 5% to about 8%, of the first particulate material. The compositions of the present invention preferably comprise from about 0.5% to about 10%, or from about 1% to about 5%, of the second particulate material. The compositions of the present invention preferably comprise from about 0.5% to about 10%, or from about 1% to about 5%, of the third particulate material. When present, the compositions of the present invention may comprise from about 0.01% to about 5%, or from about 0.1% to about 3%, of the fourth particulate material.

Non-limiting examples of suitable powders include inorganic powders (e.g., iron oxides, titanium dioxides, zinc oxides, silica), organic powders, composite powders, optical brightener particles, and mixtures of any of the foregoing. Typically, the particulate materials are free-flowing and solid (i.e., the particles are not hollow). These particulates can, for instance, be surface coated or uncoated; porous or non-porous; charged or uncharged; and can be added to the current compositions as a powder or as a pre-dispersion. In one embodiment, the particulate material is hydrophobically coated.

Suitable organic powder particulate materials include, but are not limited to, polymeric particles chosen from polymethylsilsesquioxane resin (e.g., Tospearl™ 145A available from GE Toshiba Silicone Co., Ltd.); polyamide (e.g., nylon-12 and Orgasol™ 2002D Nat C05 available from Atofina; nylon-12 available as ROPEARL from Kobo Products, Inc.); polyethylenes (e.g., Microthene™ FN510-00 available from Equistar and Micropoly® 220L from Micro Powders, Inc.); crosslinked polydimethylsiloxanes, e.g., Trefil™ E 506C or Trefil™ E 505C (Dow Corning Toray Silicone); polyacrylates (e.g., ethylene acrylate copolymer, sold under the name FloBead™ EA209 available from Kobo); polymethacrylates (PMMA) (e.g., Micropearl™ M 100 available from Seppic); polystyrene (e.g., Dynospheres™ available from Dyno Particles); polypropylene (e.g., Mattewax™ 511 from Micro Powders, Inc); polytetrafluoroethylene (PTFE); polyurethanes; starch and starch derivatives, e.g., aluminum starch octenylsuccinate (available as Dry Flo™ from National Starch); silicone resin; platelet shaped powder made from L-lauroyl lysine; composite particles; and mixtures thereof. Copolymers derived from monomers of the aforementioned materials can also be used. The aforementioned polymers derived from carboxylic acid containing monomer further include ester and salts of the monomers. Inorganic materials include but are not limited to silica. In one embodiment, the first particulate material is polyolefin. In one embodiment, the second particulate material is aluminum starch octenylsuccinate. In one embodiment, the third particulate material is polymethylsilsesquioxane. In one embodiment, a fourth particulate material is polyamide. In another embodiment, an additional particulate is titanium dioxide.

D. Thickening Agent

The composition of the present invention may include one or more thickening agents. The composition of the present invention may comprise from about 0.1% to about 5%, or, alternatively, from about 1% to about 3%, of a thickening agent when present. Suitable classes of thickening agents include but are not limited to carboxylic acid polymers, polyacrylamide polymers, sulfonated polymers, copolymers thereof, hydrophobically modified derivatives thereof, and mixtures thereof.

Suitable thickening agents include carboxylic acid polymers such as the carbomers (e.g., the CARBOPOL® 900 series such as CARBOPOL® 954). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic Corp. and Viscolam At 100 P available from Lamberti S.p.A. (Gallarate, Italy). Another commercially available material comprising a sulfonated polymer is Sepiplus 400 available from Seppic Corp.

E. Humectant

The composition of the present invention may include one or more humectants. The composition of the present invention may comprise from about 3% to about 25%, alternatively, from about 5% to about 15%, of the humectant, when present. An exemplary class of humectants is polyhydric alcohols. Suitable polyhydric alcohols include polyalkylene glycols and alkylene polyols and their derivatives, including propylene glycol, dipropylene glycol, polypropylene glycol, polyethylene glycol and derivatives thereof; sorbitol; hydroxypropyl sorbitol; erythritol; threitol; pentaerythritol; xylitol; glucitol; mannitol; hexylene glycol; butylene glycol (e.g., 1,3-butylene glycol); pentylene glycol; hexane triol (e.g., 1,2,6-hexanetriol); glycerin; ethoxylated glycerine; and propoxylated glycerine.

Other suitable humectants include sodium 2-pyrrolidone-5-carboxylate, guanidine; glycolic acid and glycolate salts (e.g., ammonium and quaternary alkyl ammonium); lactic acid and lactate salts (e.g., ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; panthenol; sodium pyroglutamate (NaPCA), water-soluble glyceryl poly(meth)acrylate lubricants (such as Hispagel®) and mixtures thereof.

F. Peptide

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT), palmitoyl-lysine-threonine-threonine-lysine-serine (pal-KTTKS, available in a composition known as MATRIXYL®), palmitoyl pentapeptide-3 (available in a composition known as PROMATRIXYL®), palmitoyl-glycine-glutamine-proline-arginine (pal-GQPR, available in a composition known as) RIGIN®, and palmitoyl-oligopeptide (available in a composition known as BIOPEPTIDE CL), these being available from Sederma, France, acetyl-glutamate-glutamate-methionine-glutamine-arginine-arginine (Ac-EEMQRR; Argireline®), hexapeptide-11 (available in a composition known as PEPTAMIDE 6 AL04 from Arch Chemical), and Cu-histidine-glycine-glycine (Cu—HGG, also known as) IAMIN®. The compositions may comprise from about 1×10⁻⁷% to about 20%, alternatively from about 1×10⁻⁶% to about 10%, and alternatively from about 1×10⁻⁵% to about 5% of the peptide.

G. Opacifying Particulate Material

In certain embodiments, the personal-care composition comprises no more than about 5% of opacifying particulate material. In other embodiments, the personal-care composition comprises no more than about 3% or about 1% of opacifying particulate material. In certain embodiments of the present invention, the personal-care composition is substantially devoid (i.e., less than 0.1%) of opacifying particulate material. An “opacifying particulate material” refers to a non-fluid material having a refractive index of greater than 1.9. Refractive index can be determined by conventional methods. While not wishing to be bound in theory, limiting or eliminating the amount opacifying particulate material provides a personal-care composition that achieves a visible acute and/or chronic benefit by a mechanism other than by merely “masking” the skin or other keratinous tissue. In other words, the resulting personal-care composition does not act as a make-up that is traditionally designed to merely conceal signs of skin aging and other skin imperfections.

Examples of opacifying particulate materials are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), under the chemical class of “Opacifying Agents.” Exemplary opacifying particulate materials include titanium dioxide, zinc oxide, zirconium dioxide, iron oxides, pigments, and the like.

H. Active

The composition of the present invention may comprise at least one skin care active, useful for regulating and/or improving the condition of mammalian skin. The active may be soluble in oil or water, and may be present primarily in the oil phase and/or in the aqueous phase. Solubility in water and oil is within the knowledge of one of skill in the art, and can be determined using known methods of analysis. One of skill in the art further will understand that solubility may be affected by the type and concentration of other components in the composition, and other conditions such as pH, ionic strength, etc. Many skin care actives may provide more than one benefit, or operate via more than one mode of action; therefore, classifications herein are made for the sake of convenience and are not intended to limit the active to that particular application or applications listed. Suitable actives include, but are not limited to, vitamins, peptides, sugar amines, sunscreens, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, and antifungals.

The compositions of the present invention may comprise from about 0.001% to about 10%, alternatively from about 0.01% to about 5%, of at least one vitamin. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

The compositions of the subject invention may comprise one or more sunscreen actives (or sunscreen agents) and/or ultraviolet light absorbers. Herein, suitable sunscreen actives include oil-soluble sunscreens, insoluble sunscreens, and water-soluble sunscreens. In certain embodiments, the composition may comprise from about 1% to about 20%, or, alternatively, from about 2% to about 10%, by weight of the composition, of the sunscreen active and/or ultraviolet light absorber. Exact amounts will vary depending upon the chosen sunscreen active and/or ultraviolet light absorber and the desired Sun Protection Factor (SPF), and are within the knowledge and judgment of one of skill in the art.

Non-limiting examples of suitable oil-soluble sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof.

Non-limiting examples of suitable insoluble sunscreens include methylene bis-benzotriazolyl tetramethylbutyl-phenol, titanium dioxide, zinc cerium oxide, zinc oxide, and derivatives and mixtures thereof.

Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof.

The compositions of the present invention may comprise one or more compounds for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions of the present invention may comprise a tanning active. A suitable tanning active includes dihydroxyacetone, which is also known as DHA or 1,3-dihydroxy-2-propanone. The compositions of the present invention may comprise a safe and effective amount of one or more anti-acne actives. Examples of useful anti-acne actives include resorcinol, sulfur, salicylic acid, erythromycin, zinc, etc. Suitable anti-acne actives are described in further detail in U.S. Pat. No. 5,607,980.

The compositions of the present invention may comprise a safe and effective amount of a desquamation active. Desquamation actives enhance the skin appearance benefits of the present invention. For example, the desquamation actives tend to improve the texture of the skin (e.g., smoothness). A suitable desquamation system comprises sulfhydryl compounds and zwitterionic surfactants and is described in U.S. Pat. No. 5,681,852. Another suitable desquamation system comprises salicylic acid and zwitterionic surfactants and is described in U.S. Pat. No. 5,652,228.

The compositions of the present invention may comprise a safe and effective amount of an anti-cellulite agent. Suitable agents may include, but are not limited to, xanthine compounds (e.g., caffeine, theophylline, theobromine, and aminophylline).

The compositions of the present invention may comprise a safe and effective amount of a chelating agent. Exemplary chelators are disclosed in U.S. Pat. No. 5,487,884; International Publication No. WO91/16035; and International Publication No. WO91/16034. A suitable chelator is furildioxime and derivatives.

The compositions of the present invention may comprise a skin lightening agent. Suitable skin lightening agents include kojic acid, arbutin, tranexamic acid, ascorbic acid and derivatives (e.g., magnesium ascorbyl phosphate or sodium ascorbyl phosphate or other salts of ascorbyl phosphate), ascorbyl glucoside, and the like. Other suitable skin lightening materials include undecylenoyl phenylalanine (Sepiwhite® from SEPPIC), aloesin, Actiwhite® (Cognis), and Emblica® (Rona).

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773.

The compositions of the present invention may comprise protease inhibitors including, but are not limited to, hexamidine (including salts and derivates thereof), vanillin acetate, menthyl anthranilate, soybean trypsin inhibitor, Bowman-Birk inhibitor, and mixtures thereof.

Other suitable actives include, but are not limited to, N-acyl amino acid compounds, tanning actives, anti-acne actives, desquamation actives, anti-cellulite actives, chelating agents, skin lightening agents, flavonoids, protease inhibitors, tyrosinase inhibitors, non-vitamin antioxidants and radical scavengers, preservatives, hair growth regulators, anti-wrinkle actives, anti-atrophy actives, minerals, phytosterols and/or plant hormones, anti-inflammatory agents, antimicrobials, and antifungals. Further suitable actives include caffeine; tea extracts, e.g. white tea extract and green tea extract; ginseng; cucumber extract; rosehip oil; date palm kernel extract; witch hazel extract; dill extract; tetrahydrocurcmin; turmerone; and other natural or botanical compounds. Many of these actives are provided in further detail in U.S. Application Publication Nos. US2006/0275237A1, US2004/0175347A1, and US2006/0263309A1.

### II. Methods of Use

The present invention describes a method of regulating and/or improving the condition of mammalian skin. The method comprises the step of topically applying to mammalian skin a personal-care composition described herein. The composition may be applied to any keratinous tissue, including keratinous tissue in need of one or more benefits.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices.

The amount of the composition applied, the frequency of application, and the period of use will vary widely depending upon the level of components of a given composition and the level of regulation desired. In one embodiment, the composition is applied at least once daily, where “daily” mean a 24-hour period. The composition further may be applied as part of a treatment regimen, for example, once daily for 30 consecutive days, for 14 consecutive days, for 7 consecutive days, or, alternatively, for 2 consecutive days.

### III. Examples

| | Ex. 1 | Ex. 2 | Ex. 3 | Ex. 4 | Ex. 5 | Ex. 6 |
|---|---|---|---|---|---|---|
| Water Phase: | | | | | | |
| Water | qs | qs | qs | qs | qs | qs |
| Glycerin | 11 | 10 | 10 | 15 | 10 | 10 |
| Niacinamide | 4 | 5 | 5 | 6 | 5 | 5 |
| Allantoin | — | 0.2 | 0.2 | 0.5 | 0.2 | 0.2 |
| Disodium EDTA | 0.02 | 0.05 | 0.05 | — | 0.05 | 0.05 |
| D-panthenol | 0.7 | 1 | 1 | — | 1 | 1 |
| Carnosine | — | 0.2 | 0.2 | 0.4 | 0.2 | 0.2 |
| Green Tea Extract | — | 0.5 | 0.5 | 0.3 | 0.5 | 0.5 |
| Cucumber Extract | 1.5 | — | — | — | — | — |
| Witch Hazel Extract | — | — | — | 0.5 | — | — |
| Glydant Plus | 0.2 | 0.3 | 0.3 | 0.5 | 0.3 | 0.3 |
| Liquid¹ | | | | | | |
| Promatrixyl² | 0.26 | 0.353 | 0.353 | — | 0.353 | 0.353 |
| Biopeptide CL³ | — | 4 | — | 2 | 4 | — |
| Peptamide 6 AL04⁴ | — | — | 0.45 | 3 | — | 0.45 |
| Propylene Glycol | 2 | — | 3.55 | 6.5 | — | 3.55 |
| Triethanolamine | 0.09 | — | — | — | — | — |
| Caffeine | — | — | — | 1 | — | — |
| Polysorbate 20 | 0.8 | — | — | — | — | — |
| Aloe Vera | — | — | — | 0.15 | — | — |
| Benzyl alcohol | — | — | — | 0.4 | — | — |
| Silicone/Oil Phase: | | | | | | |
| Dimethicone 50 csk | 6 | 1 | 1 | 4 | 1 | 1 |
| Dow Corning 9045⁵ | — | 20 | 20 | 15 | 20 | 20 |
| SF-1202 Silicone | 15 | 10 | 10 | 7 | 10 | 10 |
| Fluid | | | | | | |
| Vitamin E Acetate | 0.2 | 0.5 | 0.5 | 1 | 0.5 | 0.5 |
| Laureth-4 | — | 0.2 | 0.2 | 0.6 | 0.2 | 0.2 |
| Dow Corning 1503⁶ | 3 | — | — | 3.5 | — | — |
| Dow Corning 9040⁷ | 10 | — | — | 5 | — | — |
| Isopropyl lauroyl | — | — | — | 2 | — | — |
| Sarcosinate | | | | | | |
| Undecylenoyl | 0.2 | — | — | — | — | — |
| Phenylalanine | | | | | | |
| Ethylparaben | 0.1 | — | — | 0.1 | — | — |
| Propylparaben | — | — | 0.1 | 0.1 | — | — |
| Thickener: | | | | | | |
| Sepigel 305⁸ | 1.6 | 2 | 2 | 2 | 2 | 2 |
| Simulgel EG⁹ | — | — | — | — | 2 | 3 |
| Additional | | | | | | |
| Ingredients: | | | | | | |
| Microthene | 9 | 6.5 | 6.5 | 6.5 | 6.5 | 6.5 |
| FN510-00¹⁰ | | | | | | |
| Tospearl 145A¹¹ | — | 2 | 2 | 2 | 1 | 1 |
| Ropearl 4000¹² | — | 1 | 1 | 1 | — | — |
| Dry Flo Pure¹³ | — | 1 | 1 | 1 | 3 | 3 |
| GLW65KTAP¹⁴ | — | 0.05 | 0.05 | 0.05 | 0.05 | 0.05 |
| Fragrance | — | 0.06 | 0.06 | 0.06 | 0.06 | 0.06 |
| Total: | 100% | 100% | 100% | 100% | 100% | 100% |

¹DMDM Hydantoin and Iodopropynyl Butylcarbamate blend available from Lonza, Inc.
²Palmitoyl-pentapeptide-3 blend available from Sederma
³Palmitoyl-oligopeptide blend available from Sederma
⁴Hexapeptide-11 blend available from Arch Chemical
⁵Dimethicone Crosspolymer in Cyclomethicone from Dow Corning Corporation
⁶Dimethicone/Dimethiconol blend from Dow Corning Corporation
⁷Dimethicone Crosspolymer in Cyclomethicone from Dow Corning Corporation
⁸Polyacrylamde, C13-14 Isoparaffin, and Laureth-7 from Seppic
⁹Sodium Acrylate/Sodium Acryloyldimethyl Taurate Copolymer, Isohexadecane, and Polysorbate 80 from Seppic
¹⁰Polyethylene Homopolymer particles from Equistar
¹¹Polymethylsilsesquioxane particles from GE Toshiba Silicone Company Ltd.
¹²Nylon-12 from Kobo Products, Inc.
¹³Aluminum Starch Octenylsuccinate particles from National Starch and Chemical Company
¹⁴65% aqueous titanium dioxide dispersion from Kobo Products, Inc.

In a suitable vessel, the water phase ingredients are combined and mixed until uniform; the water phase may be warmed to dissolve all ingredients. In a separate suitable container, the silicone/oil phase ingredients are combined and mixed until uniform; the silicone/oil phase may be warmed to dissolve all ingredients. Half the thickener and then the silicone/oil phase is added to the water phase and the resulting emulsion is milled (e.g., with a Tekmar T-25). The remainder of the thickener and then the remaining ingredients are then added to the emulsion while stirring. Once the composition is uniform, the product is poured into suitable containers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal-care composition in the form of a oil-in-water emulsion comprising:
a) from about 0.5% to about 30% of a silicone elastomer;
b) from about 0.5% to about 20% of a silicone fluid;
c) from about 1% to about 15% of a first particulate material;
d) from about 0.5% to about 10% of a second particulate material;
e) from about 0.5% to about 10% of a third particulate material;
f) from about 0.1% to about 5% of a polymeric thickener; and
g) water.

2. The composition of claim 1 wherein the composition comprises from about 1% to about 20% of the silicone elastomer.

3. The composition of claim 1 wherein the silicone elastomer is a non-emulsifying silicone elastomer.

4. The composition of claim 1 wherein the composition comprises from about 1% to about 15% of the silicone fluid.

5. The composition of claim 1 wherein the silicone fluid is a volatile silicone.

6. The composition of claim 1 wherein the silicone fluid is selected from a group consisting of polyalkylsiloxanes, cyclomethicones, and combinations thereof.

7. The composition of claim 1 wherein the composition comprises from about 5% to about 8% of the first particulate material.

8. The composition of claim 1 wherein the first particulate material has a median particle size of from about 5 μm to about 40 μm.

9. The composition of claim 1 wherein the first particulate material has a median particle size of from about 10 μm to about 30 μm.

10. The composition of claim 1 wherein the first particulate material is selected from a group consisting of polyethylenes, polymethylsilsesquioxanes, siloxanes, polyamides, polyolefins, polystyrenes, polytetrafluoroethylenes, polyurethanes, polymethacrylates, starch and starch derivatives, composite particles, copolymers thereof, and combinations thereof.

11. The composition of claim 1 wherein the first particulate material is polyethylene.

12. The composition of claim 1 wherein the composition comprises from about 1% to about 5% of the second particulate material.

13. The composition of claim 1 wherein the second particulate material has a median particle size of from about 0.1 μm to about 20 μm.

14. The composition of claim 1 wherein the second particulate material has a median particle size of from about 0.5 μm to about 15 μm.

15. The composition of claim 1 wherein the second particulate material is selected from a group consisting of polyethylenes, polymethylsilsesquioxanes, siloxanes, polyamides, polyolefins, polystyrenes, polytetrafluoroethylenes, polyurethanes, polymethacrylates, starch and starch derivatives, composite particles, copolymers thereof, and combinations thereof.

16. The composition of claim 1 wherein the second particulate material is a starch.

17. The composition of claim 1 wherein the composition comprises from about 1% to about 5% of the third particulate material.

18. The composition of claim 1 wherein the third particulate material has a median particle size of from about 0.1 μm to about 15 μm.

19. The composition of claim 1 wherein the third particulate material has a median particle size of from about 0.5 μm to about 10 μm.

20. The composition of claim 1 wherein the third particulate material is selected from a group consisting of polyethylenes, polymethylsilsesquioxanes, siloxanes, polyamides, polyolefins, polystyrenes, polytetrafluoroethylenes, polyurethanes, polymethacrylates, starch and starch derivatives, composite particles, copolymers thereof, and combinations thereof.

21. The composition of claim 1 wherein the third particulate material is polymethylsilsesquioxane.

22. The composition of claim 1, further comprising from about 0.01% to about 5% of a fourth particulate material.

23. The composition of claim 1 wherein the composition comprises from about 0.1% to about 3% of the fourth particulate material.

24. The composition of claim 1 wherein the fourth particulate material has a median particle size of from about 1 μm to about 25 μm.

25. The composition of claim 1 wherein the fourth particulate material has a median particle size of from about 3 μm to about 20 μm.

26. The composition of claim 1 wherein the fourth particulate material is selected from a group consisting of polyethylenes, polymethylsilsesquioxanes, siloxanes, polyamides, polyolefins, polystyrenes, polytetrafluoroethylenes, polyurethanes, polymethacrylates, starch and starch derivatives, composite particles, copolymers thereof, and combinations thereof.

27. The composition of claim 1 wherein the fourth particulate material is polyamide.

28. The composition of claim 1 wherein the composition comprises from about 1% to about 3% of a polymeric thickener.

29. The composition of claim 1 wherein the polymeric thickener is selected from a group consisting of carboxylic acid polymers, polyacrylamide polymers, sulfonated polymers, copolymers thereof, hydrophobically modified derivatives thereof, and mixtures thereof.

30. The composition of claim 1 wherein the composition further comprises from about 3% to about 25% of a humectant.

31. The composition of claim 30 wherein the composition comprises from about 5% to about 15% of a humectant.

32. The composition of claim 30 wherein the humectant is low molecular weight polyol.

33. The composition of claim 32 wherein said low molecular weight polyol is glycerin.

34. The composition of claim 1 wherein the composition further comprises a skin active selected from a group consisting of vitamins, peptides, sugar amines, sunscreens, oil control agents, non-vitamin antioxidants and radical scavengers, hair growth regulators, flavonoids, minerals, phytosterols, plant hormones, protease inhibitors, tyrosinase inhibitors, anti-inflammatory agents, N-acyl amino acid compounds, and combinations thereof.

35. The composition of claim 1 wherein the skin active is a vitamin B compound, a vitamin C compound, N-acetyl glucosamine, hexamidine, green tea extract, or combinations thereof.

36. The composition of claim 1 wherein said composition comprises no more than about 5% of an opacifying particulate material.

37. The composition of claim 1 wherein said composition comprises no more than about 1% of an opacifying particulate material.

38. The composition of claim 1 wherein said composition is substantially devoid of an opacifying particulate material.

39. The composition of claim 1 wherein the composition comprises from about 8% to about 74% of water.

40. The composition of claim 1 wherein the composition comprise from about 20% to about 50% of water.
